(19) United States
(12) Patent Application Publication
(10) Pub. No.: US 2006/0135523 A1
(43) Pub. Date: Jun. 22, 2006

(54) 2-substituted 5,6-diaryl-pyrazine derivatives as cb1 modulator

(75) Inventors: Leifeng Cheng, Molndal (SE); Thomas Elebring, Molndal (SE); Henrik Sorensen, Molndal (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/561,060

(22) PCT Filed: Jun. 16, 2004
(86) PCT No.: PCT/SE04/00969

§ 371 Date: Dec. 16, 2005

(30) Foreign Application Priority Data
Jun. 18, 2003 (GB) 0314059.7
Jun. 18, 2003 (GB) 0314061.3

(51) Int. Cl. A61K 31/501; A61K 31/4965; C07D 413/02; C07D 403/02; A61K 31/506

(52) U.S. Cl. 514/252.02; 514/255.05; 514/255.06; 544/405; 544/406; 544/238; 544/295

(57) ABSTRACT

The present invention relates to 2-substituted-5,6-diarylpyrazine compounds and their compositions, processes for their preparation, and their use in the treatment of obesity and psychiatric and neurological disorders.

### FIELD OF INVENTION

The present invention relates to certain pyrazine compounds of formula I, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WO01/70700 and EP 656354). However, there is a need for CB₁ modulators with improved physicochemical properties and/or DMPK properties and/or pharmacodynamic properties.

Pyrazinecarboxamides are reported to possess antithrombotic properties (WO 92/02513). The compounds disclosed in this document are disclaimed from the compound claims of the present invention. 5,6-Diphenyl-2-pyrazinecarboxylic acid is disclosed in CH 458 361.

Co-pending application PCT/GB02/05742 discloses compounds of the general formula (I)

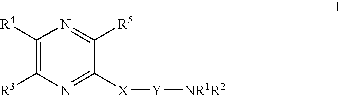

and pharmaceutically acceptable salts, prodrugs, solvates and crystalline forms thereof, in which

R¹ and R² independently represent:

a C₁₋₆alkyl group;

an (amino)C₁₋₄alkyl-group in which the amino is optionally substituted by one or more C₁₋₃alkyl groups;

an optionally substituted non-aromatic C₃₋₁₅carbocyclic group;

a (C₃₋₁₂cycloalkyl)C₁₋₃alkyl-group;

a group —(CH₂)ᵣ(phenyl)ₓ in which r is 0, 1, 2, 3 or 4, s is 1 when r is 0 otherwise s is 1 or 2 and the phenyl groups are optionally independently substituted by one, two or three groups represented by Z;

naphthyl;

anthracenyl;

a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;

1-adamantylmethyl;

a group —(CH₂)ₜ Het in which t is 0, 1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo;

or R¹ represents H and R² is as defined above;

or R¹ and R² together with the nitrogen atom to which they are attached represent a saturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy or benzyl;

X is CO or SO₂;

Y is absent or represents NH optionally substitututed by a C₁₋₃alkyl group;

R³ and R⁴ independently represent phenyl, thienyl or pyridyl each of which is optionally substituted by one, two or three groups represented by Z;

Z represents a C₁₋₃alkyl group, a C₁₋₃alkoxy group, hydroxy, halo, trifluoromethyl, trifluoromethylthio, trifluoromethoxy, trifluoromethylsulphonyl, nitro, amino, mono or di C₁₋₃alkylamino, mono or di C₁₋₃alkylamido, C₁₋₃alkylsulphonyl, C₁₋₃alkoxycarbonyl, carboxy, cyano, carbamoyl, mono or di C₁₋₃alkyl carbamoyl, sulphamoyl and acetyl; and

R⁵ is H, a C₁₋₃alkyl group, a C₁₋₃alkoxymethyl group, trifluoromethyl, a hydroxyl C₁₋₃alkyl group, C₁₋₃alkoxycarbonyl, carboxy, cyano, carbamoyl, mono or di C₁₋₃alkylcarbamoyl, acetyl, or hydrazinocarbonyl of formula —CONHNR^{a}R^{b} wherein R^{a} and R^{b} are as previously defined for R¹ and R² respectively;

with the proviso that when R¹ and R² together with the nitrogen atom to which they are attached represent 4-methylpiperazin-1-yl or R¹ represents H and R² represents methyl or 1-benzylpiperidin-4-yl; X is CO; Y is absent and R⁵ is H; then R³ and R⁴ do not both represent 4-methoxyphenyl; and their use in the treatment of obesity, psychiatric and neurological disorders.

### DESCRIPTION OF THE INVENTION

The invention relates to a compound of formula (I)

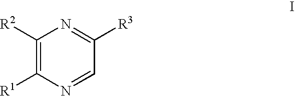

wherein R¹ and R² independently represent phenyl, thienyl or pyridyl each of which is independently optionally substituted by one or more groups represented by Z;

Z represents a C₁₋₈alkyl group, a C₁₋₃alkoxy group, hydroxy, halo, trifluoromethyl, trifluoromethylthio, trifluoromethoxy, trifluoromethylsulphonyl, nitro, mono or di C₁₋₃alkylamido, C₁₋₃alkylsulphonyl, C₁₋₃alkylsulphonyloxy, C₁₋₃alkoxycarbonyl, carboxy, cyano, carbamoyl, mono or di C₁₋₃alkyl carbamoyl, sulphamoyl, acetyl, an aromatic heterocyclic group, optionally substituted by halo, alkyl, trifluoromethyl or trifluoromethoxy and a saturated or partially unsaturated 5 to 8 membered heterocyclic group containing one or more heteroatoms selected from nitrogen, oxygen or sulphur wherein the heterocyclic group is optionally substituted by one or more C₁₋₃alkyl groups, hydroxy, fluoro, benzyl or an amino group —NR^{x}R^{y} in which R^{x} and R^{y} independently represent H or C₁₋₄-alkyl;

R³ represents a group of formula (CH₂)ₙCOOR⁷

in which n is 0, 1, 2, 3 or 4 and R⁷ represents a C₄₋₁₂alkyl group, a C₃₋₁₂cycloalkyl group or a (C₃-₁₂cycloalkyl)C₁₋₃alkyl-group each of which is optionally substituted by one or more of the following: a C₁₋₆alkyl group; fluoro, amino or hydroxy, or

R⁷ represents a group -CH₂)ₐphenyl in which a is 0, 1, 2, 3 or 4 and the phenyl group is optionally substituted by one or more groups represented by Z which may be the same or different or

R⁷ represents a saturated or partially unsaturated 5 to 8 membered heterocyclic group containing one or more of the of the following: oxygen, sulphur or nitrogen; wherein the heterocyclic group is optionally substituted by one or more C₁₋₃alkyl groups, C₁₋₃acyl groups, hydroxy, amino or benzyl; or

R³ represents a group of formula -(CH₂)ₒ—O—(CH₂)p- R⁸ in which o represents an integer 1, 2, 3 or 4 and p represents an integer 0, 1, 2, 3 or 4 and R⁸ represents a C₁₋₁₂alkyl group optionally substituted by one or more of the following: a C₁₋₆alkyl group; fluoro, hydroxy, or an amino group —NR^{x}R^{y} in which R^{x} and R^{y} independently represent H or C₁₋₄alkyl;

or R⁸ represents phenyl optionally independently substituted by one or more Z groups or R⁸ represents an aromatic heterocyclic group or a saturated or partially unsaturated 5 to 8 membered heterocyclic group containing one or more of one following: oxygen, sulphur or nitrogen wherein each of these rings is optionally substituted by one or more groups represented by Z which may be the same or different;

R³ represents a group of formula —(CH₂)_{q}R⁹ in which q is 2, 3 or 4 and R⁹ represents a C₃₋₁₂cycloalkyl group, phenyl, an aromatic heterocyclic group or a saturated or partially unsaturated 5 to 8 membered heterocyclic group containing one or more of one following: oxygen, sulphur or nitrogen wherein each of these rings is optionally substituted by one or more groups represented by Z which may be the same or different; or

R³ represents a group of formula —(CH₂)ₘ—O—(CO)—R¹⁰ in which m represents an integer 0, 1, 2, 3 or 4, and in which R¹⁰ represents a Cl 12alkyl group optionally substituted by one or more fluoro, hydroxy, or amino or R¹⁰ represents a group of formula —(CH₂)_{q}R⁹ in which q and R⁹ are as previously described; or

R³ has the following formula:

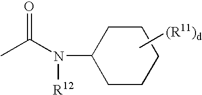

R¹¹ represents hydroxy, fluoro, carboxy, a C₁₋₆alkoxycarbonyl group or an amino group —NR^{x}R^{y} in which R^{x} and R^{y} independently represent H or C₁₋₄alkyl;

d is 1, 2 or 3, and

R¹² represents H or a C₁₋₃alkyl group, or

R³ represents a group of formula CONH—R^{z}, in which R^{z} is a piperidinyl ring substituted by a C₁₋₆alkanoyl group or R³ represents a group —COG in which G is a dihydroindole or a dihydroisoindole, linked through nitrogen to the carbonyl,

and pharmaceutically acceptable salts thereof.

It will be understood that where a substituent Z is present in more than one group that these substituents are independently selected and may be the same or different.

In another embodiment of the present invention formula, R³ has the following formula:

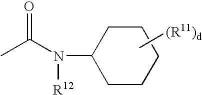

R¹¹ represents hydroxy, fluoro, carboxy, a C₁₋₆alkoxycarbonyl group or an amino group —NR^{x}R^{y} in which R^{x} and R^{y} independently represent H or C₁₋₄alkyl;

d is 1, 2 or 3,

R¹² represents H or a C₁₋₃alkyl group, and pharmaceutically acceptable salts thereof.

The term aromatic heterocyclic group means an aromatic 5-, 6-, or 7-membered monocyclic ring or a 9- or 10-membered bicyclic ring, with up to five ring heteroatoms selected from oxygen., nitrogen and sulfur. Suitable aromatic heterocyclic groups include, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl, preferably furyl, pyrrolyl, thienyl, oxazolyl, isoxazolyl, imidazolyl, pyrazolyl, oxazolyl thiazolyl, isothiazolyl, oxadiazolyl, thiadiazolyl, triazolyl, tetrazolyl, pyridyl, pyridazinyl, pyrimidinyl, pyrazinyl or 1,3,5-triazenyl and more preferably pyrrolyl, thienyl, imidazolyl, oxazolyl or pyridyl.

Suitable saturated or partially unsaturated 5 to 8 membered heterocyclic group containing one or more heteroatoms selected from nitrogen, oxygen or sulphur include, for example oxiranyl, oxetanyl, tetrahydrofuranyl, tetrahydropyranyl, 2,3-dihydro-1,3-thiazolyl, 1,3-thiazolidinyl, pyrrolinyl, pyrrolidinyl, morpholinyl, tetrahydro-1,4-thiazinyl, 1-oxotetrahydrothienyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl or tetrahydropyrimidinyl, preferably tetrahydrofuranyl, tetrahydropyranyl, pyrrolidinyl, morpholinyl, piperidinyl or piperazinyl, more preferably tetrahydrofuran-3-yl, tetrahydropyranyl, pyrrolidin-3-yl, morpholino, piperidino, piperidin-4-yl or piperazin-1-yl.

Further values of R¹, R² and R³ in compounds of formula I now follow. It will be understood that such values may be used where appropriate with any of the definitions, claims or embodiments defined hereinbefore or hereinafter.

Particularly R¹ and R² each represent phenyl independently optionally substituted by one or more chloro.

Particularly R³ represents C₄₋₁₂alkoxycarbonyl.

Particularly R³ represents a benzyloxymethyl group optionally substituted by Z in the phenyl ring of the benzyl group.

Particularly R³ represents a group C(O)O-Het wherein Het is piperidino, morpholino or pyrrolidino.

In a first group of compounds of formula I, R¹ and R² each represent 4-chlorophenyl.

In a second group of compounds of formula I, d is I and R¹¹ is hydroxyl, amino or a C₁₋₆alkoxycarbonyl group.

In a third group of compounds of formula I, d is 2 and R¹¹ is F and both fluoros are attached to the same carbon on the cyclohexyl ring.

In a fourth group of compounds of formula I, R¹² is H.

In a fifth group, the aromatic heterocyclic group is furyl, pyrrolyl, thienyl, oxazolyl, isoxazolyl, imidazolyl, pyrazolyl, oxazolyl thiazolyl, isothiazolyl, oxadiazolyl, thiadiazolyl, triazolyl, tetrazolyl, pyridyl, pyridazinyl, pyrimidinyl, pyrazinyl or 1,3,5-triazenyl.

In a sixth group, the aromatic heterocyclic group is pyrrolyl, thienyl, imidazolyl, oxazolyl or pyridyl.

In a seventh group, the saturated or partially unsaturated 5 to 8 membered heterocyclic group is tetrahydrofuranyl, tetrahydropyranyl, pyrrolidinyl, morpholinyl, piperidinyl or piperazinyl,

In a eighth group, the saturated or partially unsaturated 5 to 8 membered heterocyclic group is tetrahydrofuran-3-yl, tetrahydropyran-4-yl, pyrrolidin-3-yl, morpholino, piperidino, piperidin-4-yl or piperazin-1-yl.

“Pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of Formula I which is sufficiently basic, for example an acid-addition salt with an inorganic or organic acid such as hydrochloric, hydrobromic, sulphuric, trifluoroacetic, citric or maleic acid; or, for example a salt of a compound of Formula I which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxy-ethyl)amine.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g., chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions, which will not cause racemisation or epimerisation, or by derivatisation, with a chiral reagent. All stereoisomers are included within the scope of the invention. All tautomers, where possible, are included within the scope of the invention.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halogen” shall mean fluorine, chlorine, bromine or iodine.

Specific compounds of the invention are one or more of the following:

5,6-bis(4-chlorophenyl)-N-(cis-2-hydroxypiperidin-1-yl)pyrazine-2-carboxamide,

5,6-bis(4-chlorophenyl)-N-(trans-2-hydroxypiperidin-1-yl)pyrazine-2-carboxamide,

5,6-bis(4-chlorophenyl)-N-(4-hydroxypiperidin-1-yl)pyrazine-2-carboxamide,

5 5,6-bis(4-chlorophenyl)-N-(4,4-difluorocyclohexyl)pyrazine-2-carboxamide,

N-(1-acetylpiperidin-3-yl)-5,6-bis(4-chlorophenyl)pyrazine-2-carboxamide,

Tert-butyl 5,6-bis(4-chlorophenyl)pyrazine-2-carboxylate,

5,6-Bis (4-chlorophenyl)-pyrazine-2-yl]-(1 ,3-dihydro-isoindol-2-yl)-methanone,

2,3-bis(4-chlorophenyl)-5-{[(4-fluorobenzyl)oxy]methyl}pyrazine,

2,3- bis(4-chlorophenyl)-5-[(piperidine-1-yloxy)carbonyl]pyrazine, and pharmaceutically acceptable salts thereof.

Methods of Preparation

The compounds of the invention may be prepared as outlined in the Examples and by analogous methods. However, the invention is not limited to these methods, the compounds may also be prepared as described for structurally related compounds in the prior art.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

Pharmacological Properties

The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g., diarrhea). The compounds are also potentially useful as agents in treatment of extended abuse, addiction and/or relapse indications, e.g. treating drug (nicotine, ethanol, cocaine, opiates, etc.) dependence and/or treating drug (nicotine, ethanol, cocaine, opiates, etc) withdrawal symptoms. The compounds may also eliminate the increase in weight, which normally accompanies the cessation of smoking.

In another aspect the present invention provides a compound of formula I as previously defined for use as a medicament.

In a further aspect the present invention provides the use of a compound of formula I in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I to a patient in need thereof.

The compounds of the present invention are particulary suitable for the treatment of obesity, e.g. by reduction of appetite and body weight, maintenance of weight reduction and prevention of rebound.

Combination Therapy

The compounds of the invention may be combined with another therapeutic agent that is useful in the treatment of disorders associated with the development and progress of obesity such as hypertension, hyperlipidaemias, dyslipidaemias, diabetes and atherosclerosis. For example, a compound of the present invention may be used in combination with a compound that affects thermogenesis, lipolysis, fat absorption, satiety, or gut motility. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related is to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel

According to an additional further aspect of the present invention there is provided a combination treatment comprising the administration of an effective amount of a compound of the formula L or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;

a cholesterol absorption antagonist;

a MTP (microsomal transfer protein) inhibitor;

a nicotinic acid derivative, including slow release and combination products;

a phytosterol compound;

probucol;

an anti-coagulant;

an omega-3 fatty acid;

another anti-obesity compound;

an antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 blocker, a saluretic, a diuretic or a vasodilator;

a melanin concentrating hormone (MCH) antagonist;

a PDK inhibitor; or

modulators of nuclear receptors for example If, FXR, RXR, and RORalpha;

a SSRI;

a serotonin antagonist;

or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt thereof, in a first unit dosage form;

b) a compound from one of the other classes of compounds described in this combination section or a pharmaceutically acceptable salt, solvate, solvate of such a salt or a prodrug thereof; in a second unit dosage form; and.

c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;

b) a compound from one of the other classes of compounds described in this combination section or a pharmaceutically acceptable salt, solvate, solvate of such a salt or a prodrug thereof, in a second unit dosage form; and

c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatorheic hepatitis, osteoarthritis and some cancers) and psychiatric and neurological conditions.

Experimental Section

Abbreviations:

DCM—dichloromethane

DMF—dimethylformamide

DMAP—4-dimethylaminopyridine

EDC—1-(3-dimethylaminopropyl)-3-ethylcarbodiimide

TEA—triethylamine

TFA—trifluoroacetic acid

DMSO—dimethyl sulfoxide

DEA—Diethylamine

PCC—Pyridinium chlorochromate

PyBOP—benzotriazol-1-yl-oxytri-pyrrolidinophosphonium hexafluorophosphate

HBTU—O-Benzotriazol-1-yl-N,N,N′,N′-tetramethyluronium Hexafluorophosphate

DAST—(diethyl amino)sulphur trifluoride

DIEA—N,N-diisopropylethylamine

TBF—tetrahydrofuran

FA—formic acid

t triplet

s singlet

d doublet

q quartet

qvint quintet

m multiplet

br broad

bs broad singlet

dm doublet of multiplet

bt broad triplet

dd doublet of doublet

General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC with a mass triggered fraction collector, Shimadzu QP 8000 single quadrupole mass spectrometer equipped with 19×100 mm C8 column. The mobile phase used was, if nothing else is stated, acetonitrile and buffer (0.1 M NH₄Ac:acetonitrile 95:5).

For isolation of isomers, a Kromasil CN E9344 (250×20 mm i.d.) column was used. Heptane:ethyl acetate:DEA 95:5:0.1 was used as mobile phase (1 ml/min). Fraction collection was guided using a UV-detector (330 nm).

Purification was performed on, if nothing else is stated, a Biotage Horizon HPFC System, using prepacked columns (Si 12+M or Si 25+M). Fraction collection was guided using a UV-detector (254 nm).

Preparation of Starting Materials and Intermediates

Step A: 1,2-bis(4-chlorophenyl)-2-hydroxyethanone

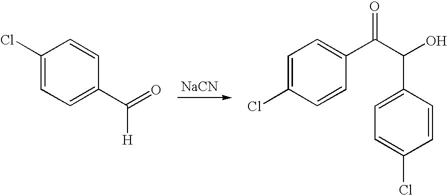

To a suspension of 4-chlorobenzaldehyde (140.6 g, 1 mol) in ethanol (130 ml) was added a solution of sodium cyanide (10.6 g, 0.216 mol) in water (105 ml). The mixture was heated at reflux for 2.5 h and then extracted with methylene chloride. The organic phase was washed with sodium bisulfite solution and the solvent was evaporated. The compound was isolated by crystallization from diethyl ether/heptan. 48 g, 34%.

¹H NMR (400 MHz) δ 7.82 (d, 2H), 7.38 (d, 2H), 7.30 (d, 2H), 7.24 (d, 2H), 5.87 (s, 1H), 4.47 (s, 1H).

MS m/z 279, 281 (M−H)⁻.

Step B: 1,2-bis(4-chlorophenyl)ethane-1,2-dione

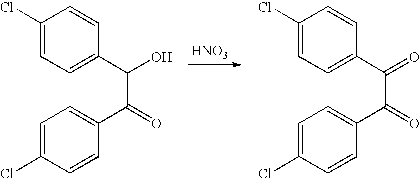

1,2-bis(4-chlorophenyl)-2-hydroxyethanone, (90 g, 0.320 mol) and nitric acid (170 ml) were heated at 100° C. until the evolution of nitrogen oxides ceased after 4 hours. The reaction mixture was cooled, and water (250 ml) was carefully added. The crude product was filtered, washed several times with water and dried under reduced pressure to give the title compound (40.4 g, 45%) as a yellow solid.

¹H NMR (500 MHz) δ 7.94 (d, 4H), 7.53 (d, 4H).

Step C: 5,6-Bis-(4-chlorophenyl) pyrazine-2-carboxylic acid

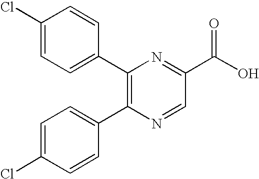

The monohydrochloride of 2,3-diaminopropionic acid (2.5 g, 17.78 mmol) and 1,2-bis(4-chlorophenyl)ethane-1,2-dione (4.965 g, 17.78 mmol), were dissolved in a solution of sodium hydroxide (3.0. g, 75 mmol) in methanol (100 ml) and refluxed for 2 hours under argon. Air was bubbled through and the reaction continued at room temperature for 20 hours. The methanol was evaporated and the product redissolved in water. Hydrochloric acid (aq, 2 M) was added until the mixture reached pH 2. The solution was extracted with diethyl ether and dried over MgSO₄. Recrystallisation from methanol gave the title compound (1.57 g, 26%).

¹H NMR (399.964 MHz) δ 9.41 (s, 1H), 7.48-7.32 (m, 8H).

MS m/z 343, 345, 347 (M−H)⁻

Step D: 5,6-bis(4-chlorophenyl)pyrazine-2-carbonyl chloride

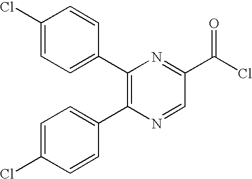

To a suspension of 5,6-bis(4-chlorophenyl)pyrazine-2-carboxylic acid, Inte. C (485 mg, 1.41 mmol) in DCM (5 ml) was added a solution of oxalyl chloride (1 ml, 7.88 mmol) in DCM (10 ml) and DMP (0.2 ml) at room temperature. The solvent and unreacted oxalyl chloride was evaporated. The crude product was used without further purification.

Step E: [5,6-bis(4-chlorophenyl)pyrazin-2-yl]methanol

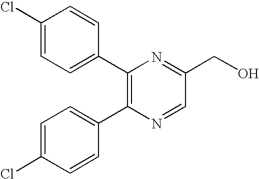

To a suspension of 5,6-bis(4-chlorophenyl)pyrazine-2-carboxylic acid, Inte. C (900 mg, 2.61 mmol) in THF (25 ml) were added ethylchloroformate (340 mg, 3.13 mmol) and DIEA (505 mg, 3.91 mmol). The mixture was stirred at room temperature for 5 hours. Methanol (2 ml) was added and then NaBH₄ (600 mg, 15.86 mmol) in small portions at 0° C. Stirring was continued at 0° C. for a further 1 h. Diethyl ether (15 ml) was added and the product was extracted with diethyl ether. The ether phase was dried over MgSO₄.

Purification by flash chromatography (SiO₂, toluene: ethyl acetate) gave the title compound (230 mg, 44%).

¹H NMR. (400 MHz) δ 8.61 (s, 1H), 7.27-7.22 (m, 4H), 7.36-7.30 (m, 4H), 4.81 (s, 2H), 4.60-4.10 (br, 1H).

³C NMR (100 MHz) δ 153.28, 150.23, 150.15, 140.45, 136.64, 136.62, 135.46, 135.33, 131.23, 129.27, 128.94, 63.16.

MS m/z 331, 333, 335 (M+H)⁺

### EXAMPLE 1

### 5,6-bis(4-chlorophenyl)1-N-(cis-2-hydroxycyclohexyl)pyrazine-2-carboxamide

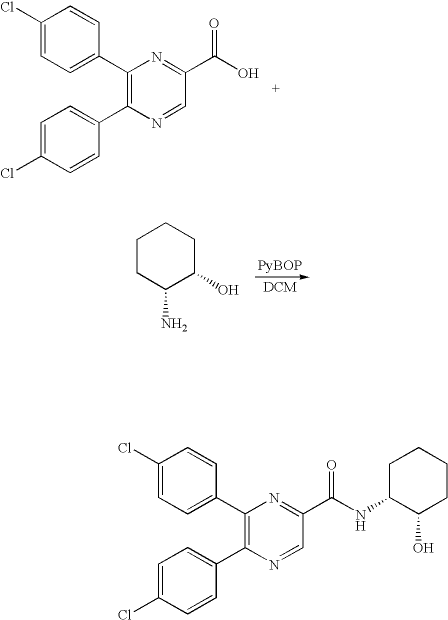

Cis-2-cyclohexanol hydrochloride (107 mg, 0.71 mmol), 5,6-bis(4-chlorophenyl)pyrazine-2-carboxylic acid (200 mg, 0.579 mmol) and TEA (0.5 ml) were dissolved in 5 ml DCM and cooled to 0° C. A solution of PyBOP(0.539 mg, 1.04 mmol) in 1 ml DCM was added dropwise. The temperature was kept at 0° C. for 15 minutes. The reaction was continued at room temperature for 3 hours. The mixture was washed with water and dried over MgSO₄.

It was purified by flash chromatography (SiO₂, gradient from 100% toluene to 100% ethyl acetate) to give the title compound (216 mg, 84%).

¹H NMR (399.964 MHz) δ 9.32 (s, 1H), 8.16 (d, 1H), 7.46-7.27 (m, 8H), 4.22-4.10 (m, 1H), 4.09-4.02 (br, 1H), 2.24-2.13 (br, 1H), 1.87-1.54 (m, 6H), 1.54-1.37 (m, 2H).

¹³C NMR (100.58 MHz) δ 162.80, 153.86, 149.46, 142.14, 141.94, 136.27, 136.03, 135.83, 131.30, 131.18, 129.10, 129.05, 69.26, 51.29, 32.11, 27.35, 23.96, 20.04.

MS m/z 442, 444, 446 (M+H)⁺.

### EXAMPLE 2

5,6-bis(4-chlorophenyl)-N-(trans-2-hydroxycyclohexyl)pyrazine-2-carboxamide

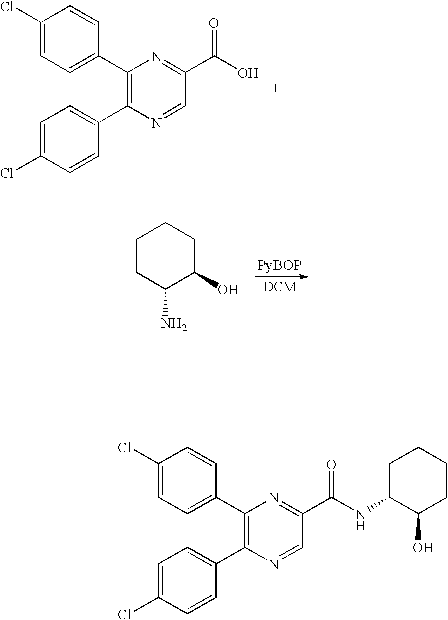

Trans-2-cyclohexanol hydrochloride (107 mg, 0.71 mmol) and 5,6-bis(4-chlorophenyl)pyrazine-2-carboxylic acid (200 mg, 0.579 mmol) were reacted as described in Example 1 to give the title compound (179 mg, 70%).

¹H NMR (399.964 MHz) δ 9.34 (s, 1H),7.79 (d, 1H), 7.44-7.26 (m, 8H), 3.95-3.80 (m, 1H), 3.55-3.43 (m, 1H), 3.34-2.79 (br, 1H), 2.20-2.00 (m, 2H), 1.87-1.66 (m, 2H), 1.50-1.18 (m, 4H).

¹³C NMR (100.58 MHz, CDCl3) δ 164.23, 154.11, 149.56, 142.14, 141.79, 136.24, 136.11, 135.89, 131.32, 131.18, 129.05, 129.14, 75.13, 56.11, 34.77, 31.76, 24.81, 24.32.

MS n/z 442, 444, 446 (M+H)⁺.

### EXAMPLE 3

### 5,6-bis(4-chlorophenyl)-N-(trans-4-hydroxycyclohexyl)pyrazine-2-carboxamide

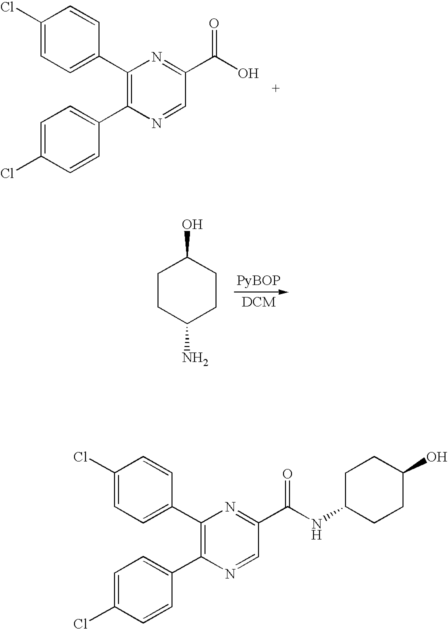

Trans-4-cyclohexanol hydrochloride (107 mg, 0.71 mmol) and 5,6-bis(4-chlorophenyl)pyrazine-2-carboxylic acid (200 mg, 0.579 mmol) were reacted as described in Example 1 to give the title compound (231 mg, 90%).

¹H NMR (399.964 MHz) δ 9.37 (s, 1H), 7.61 (d, 1H), 7.44-7.27 (m, 8H), 4.07-3.93 (m, 1H), 3.73-3.60 (m, 1H), 2.17-1.99 (m, 4H), 1.76-1.62 (br, 1H), 1.56-1.32 (m, 4H).

¹³C NMR (100.58 MHz) δ 162.41, 153.94, 149.48, 142.11, 142.11, 136.32, 136.18, 136.06, 135.87, 131.31, 131.16, 129.14, 129.06, 69.94, 48.01, 34.18, 30.98.

MS m/z 442, 444, 446 (M+H)⁺.

### EXAMPLE 4

### 5,6-bis(4-chlorophenyl)-N-(4,4-difluorocyclohexyl)pyrazine-2-carboxamide

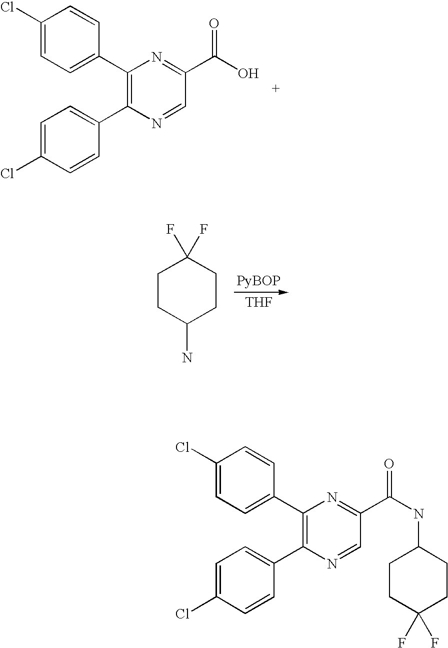

Step A (4,4-difluorocyclohexyl)amine

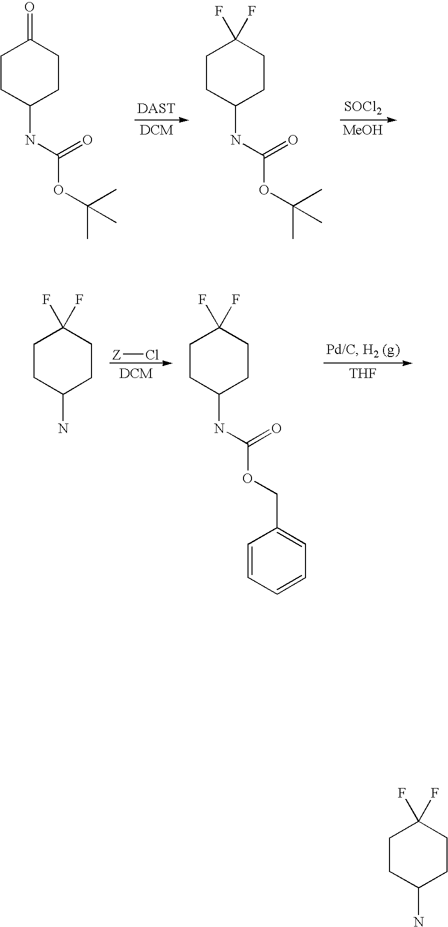

To a solution of N-4-Boc-cyclohexanone (600 mg, 2.81 mmol) in DCM (3 ml) at 0° C. was added DAST (455 mg, 2.81 mmol) dropwise. After 70 minutes the temperature was increased to room temperature and after 3 hours to reflux for 5 minutes. The solvent was removed in vacuo and the product was purified with a flash column (silica gel, toluene, 100% to EtOAc, 100%). The suspension of the Boc-protected material in methanol (5 ml) ws treated with a solution of thionyl chloride (2 ml, 27.57 mmol) in methanol (20 ml) dropwise. The reaction was continued at room temperature for 30 minutes. The solvent was evaporated in vacuo. The crude material was retaken in pyridine (5 ml) and treated with a solution of benzylchloroformate (532 mg, 3.12 mmol) in 1 ml DCM. The mixture was stirred for 58 hours. It was washed with HCl (aq) and K₂CO₃ (aq). The Z-protected compound was purified by flash chromatography (SiO₂, toluene), 212 mg (28%). The Z-group was removed by stirring under H₂ atmosphere in THF (10 ml) with palladium on activated carbon (40 mg, 10 wt %, Pd) for 4 h. It was filtered through Celite 521 and evaporated in vacuo to give a crude material.

Step B 5,6-bis(4-chlorophenyl)-N-(4,4-difluorocyclohexyl)pyrazine-2-carboxamide

(4,4-difluorocyclohexyl)amine and 5,6-bis(4-chlorophenyl)pyrazine-2-carboxylic acid (200 mg, 0.579 mmol) were reacted as described in Example 1 THP (60 ml) was used in stead of DCM. The product was purified with prepHPLC (kromasil C8 column, ammonium acetate (aq, 0.1 M):acetonitrile) to give the title compound as a white powder (116 mg, 43%).

¹H NMR (399.964 MHz) δ 9.35 (s, 1H), 7.69 (d, 1H), 7.43-7.28 (m, 8H), 4.20-4.08 (m, 1H), 2.21-2.06 (m, 4H), 2.03-1.83 (m, 2H), 1.78-1.64 (m, 2H).

¹³C NMR (100.58 MHz) o 162.55, 154.13, 149.57, 142.03, 141.80, 136.19, 136.12, 135.94, 131.30, 131.13, 129.17, 129.06, 122.53 (t), 46.61, 32.47 (t), 28.90, 28.81.

MS m/z 462, 464, 466 (M+H)⁺.

### EXAMPLE 5

Step A: Tert-butyl 3-({[5,6-bis(4-chlorophenyl)pyrazin-2-yl]carbonyl}amino)piperidine-1-carboxylate

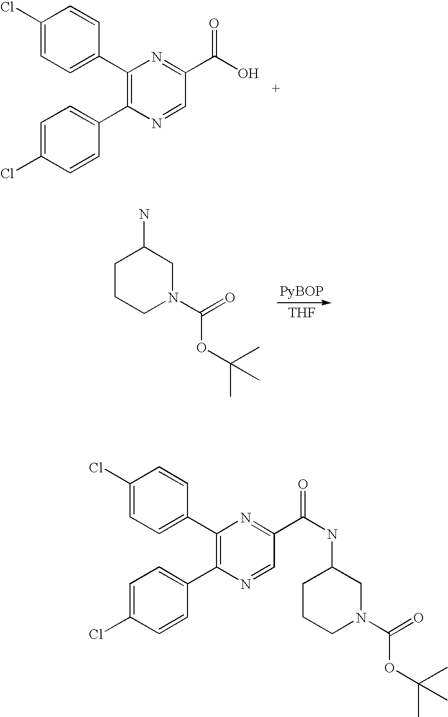

PyBOP (508 mg, 0.976 mmol), dissolved in DCM (1 ml), was added to tert-butyl 3-aminopiperidine-1-carboxylate (151 mg, 0.754 mmol), and 5,6-bis(4-chlorophenyl)pyrazine-2-carboxylic acid (200 mg, 0.579 mmol), dissolved in DCM (5 ml) and TEA (0.5 ml), at 0° C. The reaction was continued at 0° C. for 15 minutes and thereafter s at room temperature 3 hours. The solution was extraced with water and dried over MgSO₄. Finally the product was purified by flash chromatography (SiO₂, toluene:ethyl acetate 9:1) to give the subtitle compound (263 mg, 86%).

¹H NMR (400 MHz, CDCl₃) δ 9.34 (s, 1H), 8.12-7.72 (br, 1H), 7.42-7.24 (m, 8H), 4.20-4.09 (m, 1H), 3.74-3.32 (m, 4H), 1.98-1.48 (m, 4H), 1.34 (s, 9H).

¹³C NMR (100 MHz, CDCl₃) δ 162.59, 155.20, 153.94, 149.54, 141.93, 136.21, 135.99, 135.76, 131.29, 131.16, 129.20, 129.09, 128.99, 80.06, 48.39, 45.72, 43.84, 29.95, 28.48, 22.73.

MS m/z 527, 529, 531 (M+H)⁺.

Step B: 5,6-bis(4-chlorophenyl)-N-piperidin-3-ylpyrazine-2-carboxamide hydrochloride

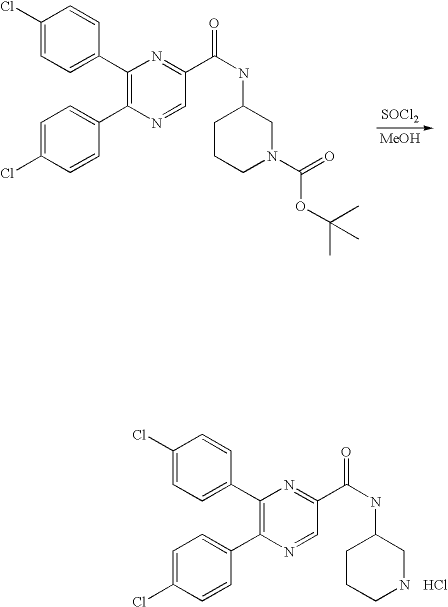

Thionyl chloride (1 ml, 13.79 mmol) dissolved in methanol (10 ml) was added drop wise to tert-butyl-3-({[5,6-bis(4chlorophenyl)pyrazin-2-yl]carbonyl}amino)piperidine-1-carboxylate (263 mg, 0.498 mmol), dissolved in 2 ml methanol. The reaction was continued at room temperature for 1 hour where after the solvent was evaporated and the product freeze dried. The subtitle compound was obtained as a white powder (230 mg, 99%).

¹H NMR (400 MHz, CDCl₃) δ 10.00-9.68 (br, 2H), 9.28 (s, 1H), 8.22 (d, 1H), 7.54-7.28 (m, 8H), 4.64-4.50 (m, 1H), 3.60-3.48 (m, 1H), 3.34-3.21 (m, 2H), 3.21-3.11 (m, 1H), 2.07-1.98 (m, 3H), 1.98-1.86 (m, 1H).

¹³C NMR (100 MHz, CDCl₃) δ 163.33, 154.19, 149.74, 141.87, 141.43, 136.13, 136.09, 135.99, 135.81, 131.55, 131.28, 129.02, 47.17, 44.11, 43.81, 28.40, 20.44.

HRMS Calcd for [C₂₂H₂₁N₄OCl₂]⁺: 427.109. Found: 427.110.

Step C: N-(1-acetyl-piperidin-3-yl)-5,6-bis(4-chlorophenyl)pyrazine-2-carboxamide

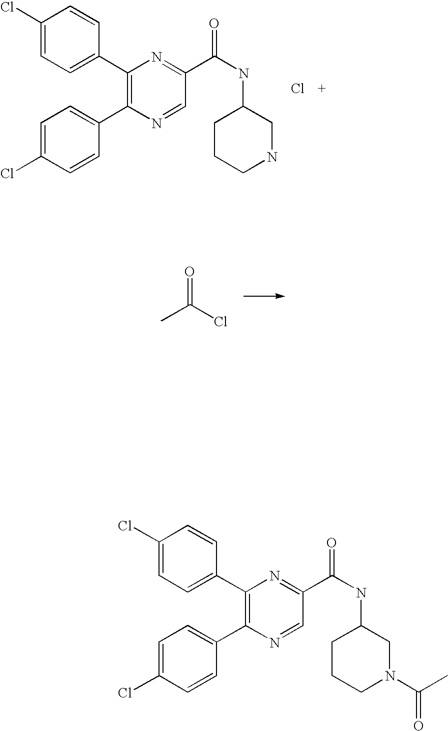

Acetylchloride (100 mg, 1.27 mmol), dissolved in 2 ml DCM was added to 5,6-Bis(4-chlorophenyl)-N-piperidin-3-ylpyrazine-2-carboxamide hydrochloride (67.0 mg, 0.145 mmol) dissolved in 3.5 ml pyridine, and reacted at room temperature 2.5 hours. Water and diethylether were added, the phases separated and the organic phase extracted with HCl (aq), K₂CO₃ (aq) and dried over MgSO₄ to give the subtitle compound as slightly yellow powder (67.0 mg, 99%).

¹H NMR (400 MHz, CDCl₃, T=25° C., rotamers)δ 9.36 and 9.32 (s, 1H); 8.02 and 7.78 (d, 1H), 7.50-7.20 (m, 8H), 4.24-4.06 (m, 1H), 3.89 and 3.76 (d, 2H), 3.52-3.40 and 3.35-3.24 (m, 2H), 2.14 and 2.11 (s, 3H), 2.12-2.02 and 2.02-1.88 (m, 2H), 1.82-1.70 and 1.70-1.58 (m, 2H).

¹³C NMR (100 MHz, CDCl₃, T=25° C., rotamers) δ 170.05, 169.68, 162.95, 162.61, 154, 36, 153.89, 149.71, 149.32, 141.88, 141.73, 141.60, 141.48, 136.28, 136.20, 136.03, 135.88, 131.30, 131.10, 129.22, 129.09, 129.05, 51.04, 47.20, 46.48, 46.33, 46.01, 41.98, 30.21, 29.85, 23.40, 23.01, 21.76, 21.71.

HRMS Calcd for [C₂₄H₂₂N₄O₂Cl₂+H]⁺: 469.120. Found: 469.119.

### EXAMPLE 6

Tert-butyl 5,6-bis(4-chlorophenyl)pyrazine-2-carboxylate

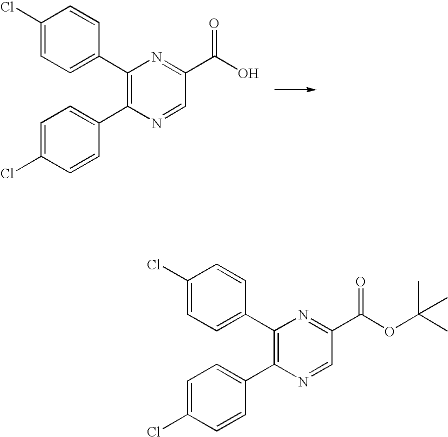

5,6-bis(4-chlorophenyl)pyrazine-2-carboxylic acid (152 mg, 0.44 mmol) was heated to 77° C. in toluene (5 ml). (Di-tert-butoxymethyl)dimethylamine (358 mg, 1.76 mmol) was added and the mixture refluxed over night (20 h). Water and diethylether were added. The phases were separated and the organic phase extracted with NaHCO₃ (aq) and water. Finally the product was purified by flash chromatography (SiO₂, toluene) to give a slightly yellow powder (92 mg, 52%).

¹H NMR (400 MHz, CDCl₃) δ 9.16 (s, 1H), 7.48-7.28 (m, 8H), 1.66 (s, 9H).

¹³C NMR (100 MHz, CDCl₃) δ 162.96, 153.53, 151.09, 143.17, 142.06, 136.29, 136.23, 136.08, 135.76, 131.41, 131.28, 129.07, 129.01, 83.45, 28.33.

HRMS Calcd for [C₂₁H₁₈N₂O₂Cl₂+H]⁺: 401.082 Found: 401.080.

### EXAMPLE 7

### 5,6-bis (4-chlorophenyl)-pyrazine-2-yl]-(1,3-dihydro-isoindol-2-yl)-methanone

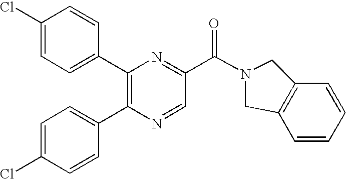

5,6-bis (4-chlorophenyl)-pyrazine-2-carboxylic acid (100 mg, 0.29 mmol), triethyl amine (0.81 ml, 20 equiv.) and isoindoline (48 mg, 1.4 equiv) were suspended in dichloromethane (8 ml) and cooled to 0° C. in an ice bath. Benzotriazol-i-yl-oxytri-pyrrolidinophosphonium hexafluorophosphate (256 mg, 1.7 equiv.) dissolved in dichloromethane (2 ml) was added dropwise and the resulting suspension was stirred at 0° C. for 15 minutes and then at room temperature for 3 hours.

The reaction mixture was diluted with dichloromethane (60 ml) and washed with water (4×20 ml) and brine (20 ml). The organic layer was dried (MgSO₄), filtered and concentrated under reduced pressure. The residue was purified by flash chromatography (ethyl acetate-heptane), which after removal of the solvent gave the product as a light yellow solid.

¹H-NMR (CDCl₃): 5.12 (s, 2H), 5.33 (s, 2 H), 7.28-7.53 (m, 12H), 9.26 (s, 1H).

MS: m/z 446 (M+H).

### EXAMPLE 8

2,3-bis(4-chlorophenyl)-5-{[(4-fluorobenzyl)oxy]methyl}pyrazine

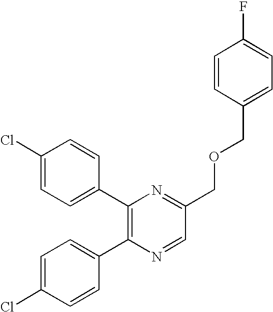

[5,6-bis(4-chlorophenyl)pyrazin-2-yl]methanol, Inte. E (230 mg, 0.69 mmol) was dissolved in DCM (3 ml) and mixed with water (2 ml). NaOH (0.53 mg, 13.25 mmol) and tetrabutylammonium hydrogen sulphate (18 mg, 0.05 mmol) were added at room temperature. 4-fluorobenzyl bromide (145 mg, 0.77 mmol) was added and the mixture stirred for 4 hours at room temperature. Diethyl ether (10 ml) was added and the product was extracted with water and dried (MgSO₄) to yield the product (285 mg, 93%).

¹H NMR (399.964 MHz) δ 8.78 (s, 1H), 7.41-7.35 (m, 6H), 7.31-7.27 (m, 4H), 7.09-7.01 (m, 2H), 4.79 (s, 2H), 4.69 (s, 2H).

### EXAMPLE 9

2,3- bis(4-chlorophenyl)-5-[(piperidine-1-yloxy)carbonyl]pyrazine

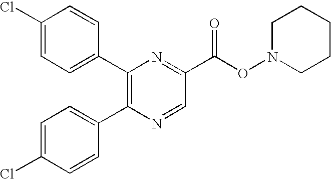

To a solution of 5,6-bis(4-chlorophenyl)pyrazine-2-carbonyl chloride, Inte. D (84 mg, 0.23 mmol) in DCM (1 ml) was added slowly at room temperature a solution of hydroxypiperidine (93 mg, 0.91 mmol) in pyridine (5 ml). After 40 minutes at room temperature, the solvent was removed in vacuo and the residue redissolved in diethyl ether. Extracted with 1M HCl (aq) and K₂CO₃ (aq) and dried (MgSO₄). The solvent was removed in vacuo to yield the subtitle compound (58 mg, 59%).

¹H NMR (399.964 MHz) δ 9.19 (s, 1H), 7.47-7.26 (m, 8H), 3.71-3.50 (m, 2H), 3.16-2.74 (m, 2H), 1.98-1.77 (m, 4H), 1.77-1.57 (m, 1H), 1.40-1.23 (m, 1H).

¹³C NMR (100.58 MHz) δ 162.59, 154.17, 151.20, 143.18, 140.79, 136.22, 136.17, 136.09, 135.89, 131.42, 131.29, 129.08, 129.04, 57.87, 25.30, 23.27.

MS m/z 428, 430, 432 (M+H)⁺.

Pharmacological Activity

Compounds of the present invention are active against the receptor product of the CB1 gene. The affinity of the compounds of the invention for central cannabinoid receptors is demonstrable in methods described in Devane et al., Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of is agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x) UD)) and the IC50 value determined as the concentration required to give half maximal inhibition of GTPγS binding under the conditions used.

The compounds of the present invention are active at the CB1 receptor (IC50<1 micromolar). Most preferred compounds have IC50<200 nanomolar.

The compounds of formula I are selected because of their superior potency in vitro and/or higher affinity, leading to better in vivo efficacy. The compounds also have a better selectivity profile, which is expected to improve in vivo safety.

In addition the compounds of the present invention may have improved DMPK (Drug Metabolism and Pharmacokinetic) properties, for example improved metabolic stability in vitro or bioavailability. The compounds also have an improved solubility and/or a promising toxicological profile.

1. A compound of formula (I): 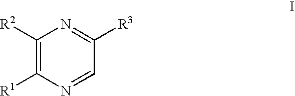
wherein R¹ and R² independently represent phenyl, thienyl or pyridyl, each of which is independently optionally substituted by one or more groups represented by Z;
Z represents a C₁₋₈alkyl group, a C₁₋₆alkoxy group, hydroxy, halo, trifluoromethyl, trifluoromethylthio, trifluoromethoxy, trifluoromethylsulphonyl, nitro, mono or di C₁₋₃alkylamido, C₁₋₃alkylsulphonyl, C₁₋₃alkylsulphonyloxy, C₁₋₃alkoxycarbonyl, carboxy, cyano, carbamoyl, mono or di C₁₋₃alkyl carbamoyl, sulphamoyl, acetyl, an aromatic heterocyclic group, optionally substituted by halo, alkyl, trifluoromethyl or trifluoromethoxy or Z represents a saturated or partially unsaturated 5- to 8-membered heterocyclic group containing one or more heteroatoms selected from nitrogen, oxygen or sulphur wherein the heterocyclic group is optionally substituted by one or more C₁₋₃alkyl, hydroxy, fluoro, benzyl or an amino group —NR^{x}R^{y} in which R^{x} and R^{y} independently represent H or C₁₋₄alkyl;
R³ represents a group of formula (CH₂)ₙCOOR⁷ in which n is 0, 1, 2, 3 or 4, and R⁷ represents a C₄₋₁₂alkyl group, a C₃₋₁₂cycloalkyl group or a (C₃₋₁₂cycloalkyl)C₁₋₃alkyl-group each of which is optionally substituted by one or more of the following: a C₁₋₆alkyl, fluoro, amino or hydroxyl group, or R⁷ represents a group —CH₂)ₐphenyl in which a is 0, 1, 2, 3 or 4, and the phenyl group is optionally substituted by one or more groups represented by Z which may be the same or different or R⁷ represents a saturated or partially unsaturated 5- to 8 membered heterocyclic group containing one or more of the following: oxygen, sulphur or nitrogen; wherein the heterocyclic group is optionally substituted by one or more C₁₋₃alkyl, C₁₋₃acyl, hydroxy, amino or benzyl groups; or
R³ represents a group of formula —(CH₂)ₚ—O—(CH₂)ₚ—R⁸ in which o represents an integer 1, 2, 3 or 4, and p represents an integer 0, 1, 2, 3 or 4, and R⁸ represents a C₁₋₁₂alkyl group optionally substituted by one or more of the following: a C₁₋₆alkyl, fluoro, hydroxy, or an amino group —NR^{x}R^{y} in which R^{x} and R^{y} independently represent H or C₁₋₄alkyl; or R⁸ represents phenyl optionally independently substituted by one or more Z groups or R⁸ represents an aromatic heterocyclic group or a saturated or partially unsaturated 5- to 8 membered heterocyclic group containing one or more of the following: oxygen, sulphur or nitrogen wherein each of these rings is optionally substituted by one or more groups represented by Z which may be the same or different; or
R³ represents a group of formula —(CH₂)_{q}R⁹ in which q is 2, 3 or 4 and R⁹ represents a C₃-₁₂cycloalkyl group, phenyl, an aromatic heterocyclic group or a saturated or partially unsaturated 5- to 8 membered heterocyclic group containing one or more of the following: oxygen, sulphur or nitrogen wherein each of these rings is optionally substituted by one or more groups represented by Z which may be the same or different; or
R³ represents a group of formula —(CH₂)ₘ—O—(CO)—R¹⁰ in which m represents an integer 0, 1, 2, 3 or 4, and in which R¹⁰ represents a C₁₋₁₂alkyl group optionally substituted by one or more fluoro, hydroxy, or amino groups or R¹⁰ represents a group of formula —(CH₂)_{q}R⁹; or
R³ has the following formula: 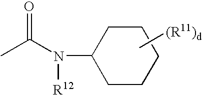
wherein R¹ i represents hydroxy, fluoro, carboxy, a C₁₋₆alkoxycarbonyl group or an amino group —NR^{x}R^{y} in which R^{x} and R^{y} independently represent H or C₁₋₄alkyl;
d is 1, 2 or 3;
R¹² represents H or a C₁₋₃alkyl group; and
R³ represents a group of formula CONH—R^{z}, in which R^{z} is a piperidinyl ring substituted by a C₁₋₆alkanoyl group or R³ represents a group —COG in which G is a dihydroindole or a dihydroisoindole, linked through nitrogen to the carbonyl,
or a pharmaceutically acceptable salt thereof.

2. A compound according to claim 1, wherein R³ has the following formula: 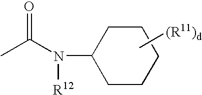
or a pharmaceutically acceptable salt thereof.

3. A compound according to claim 1, wherein R¹ and R² each represent phenyl independently optionally substituted by one or more chloro.

4. A compound according to claim 1, wherein R³ represents C₄₋₁₂alkoxycarbonyl.

5. A compound according to claim 1, wherein R³ represents a benzyloxymethyl group optionally substituted by Z in the phenyl ring of the benzyloxymethyl group.

6. A compound according to claim 1, wherein R³ represents a group C(O)O-Het wherein Het is piperidino, morpholino or pyrrolidino.

7. A compound according to claim 3, wherein R¹ and R² each represent 4-chlorophenyl.

8. A compound according to claim 2, wherein d is 1 and R¹¹ is hydroxyl, amino or a C₁₋₆alkoxycarbonyl group.

9. A compound according to claim 2, wherein d is 2 and R¹¹ is F and both occurrences of R¹¹ are attached to the same carbon.

10. A compound according to claim 2, wherein R¹² is H.

11. A compound according to claim 1, wherein the aromatic heterocyclic group is furyl, pyrrolyl, thienyl, oxazolyl, isoxazolyl, imidazolyl, pyrazolyl, oxazolyl thiazolyl, isothiazolyl, oxadiazolyl, thiadiazolyl, triazolyl, tetrazolyl, pyridyl, pyridazinyl, pyrimidinyl, pyrazinyl or 1,3,5-triazenyl.

12. A compound according to claim 1, wherein the aromatic heterocyclic group is pyrrolyl, thienyl, imidazolyl, oxazolyl or pyridyl.

13. A compound according to claim 1, wherein the saturated or partially unsaturated 5- to 8 membered heterocyclic group is tetrahydrofuranyl, tetrahydropyranyl, pyrrolidinyl, morpholinyl, piperidinyl or piperazinyl,

14. A compound according to claim 1, wherein the saturated or partially unsaturated 5- to 8 membered heterocyclic group is tetrahydrofuran-3-yl, tetrahydropyran-4-yl, pyrrolidin-3-yl, morpholino, piperidino, piperidin-4-yl or piperazin-1-yl.

15. A compound selected from:
5,6-bis(4-chlorophenyl)-N-(cis-2-hydroxypiperidin-1-yl)pyrazine-2-carboxamide,
5,6-bis(4-chlorophenyl)-N-(trans-2-hydroxypiperidin-1-yl)pyrazine-2-carboxamide,
5,6-bis(4-chlorophenyl)-N-(4-hydroxypiperidin-1-yl)pyrazine-2-carboxamide,
5,6-bis(4-chlorophenyl)-N-(4,4-difluorocyclohexyl)pyrazine-2-carboxamide,
N-(1-acetylpiperidin-3-yl)-5,6-bis(4-chlorophenyl)pyrazine-2-carboxamide,
Tert-butyl 5,6-bis(4-chlorophenyl)pyrazine-2-carboxylate,
5,6-Bis (4-chlorophenyl)-pyrazine-2-yl]-(1,3-dihydro-isoindol-2-yl)-methanone,
2,3-bis(4-chlorophenyl)-5-{[(4-fluorobenzyl)oxy]methyl}pyrazine, or
2,3- bis(4-chlorophenyl)-5-[(piperidine-1-yloxy)carbonyl]pyrazine,
or a pharmaceutically acceptable salt thereof.

16. (canceled)

17. A pharmaceutical formulation comprising a compound of claim 1 and a pharmaceutically acceptable adjuvant, diluent or carrier.

18. (canceled)

19. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound of claim 1 or a formulation of claim 17 to a patient in need thereof.

20. A method for the treatment of obesity comprising administering a pharmacologically effective amount of a compound of claim 1 or a formulation of claim 17 to a patient in need thereof.
